(19) United States
(12) Patent Application Publication
(10) Pub. No.: US 2026/0231328 A1
(43) Pub. Date: Aug. 6, 2026

(54) CIRCUIT STRUCTURE, FRONT-END ASSEMBLY, AND ENDOSCOPE

(71) Applicant: HUNAN VATHIN MEDICAL INSTRUMENT CO., LTD., Xiangtan (CN)

(72) Inventor: Zhenhua ZHOU, Xiangtan (CN)

(73) Assignee: HUNAN VATHIN MEDICAL INSTRUMENT CO., LTD., Xiangtan (CN)

(21) Appl. No.: 19/145,945

(22) PCT Filed: Jan. 12, 2024
(86) PCT No.: PCT/CN2024/071933

§ 371 Date: Jul. 7, 2025

(30) Foreign Application Priority Data
Feb. 28, 2023 (CN) 202310178509.4

(51) Int. Cl. H05K 1/14; A61B 1/00; A61B 1/005; A61B 1/05

(52) U.S. Cl. CPC H05K 1/147; A61B 1/00124; A61B 1/0055; A61B 1/0057; A61B 1/051; H05K 2201/10121

(57) ABSTRACT

A circuit structure, a front-end assembly, and an endoscope are provided. The circuit structure includes a connecting unit and a mounting unit connected to a distal end of the connecting unit, where the connecting unit includes a first extension segment, a bending segment, and a second extension segment connected sequentially; an extension direction of the first extension segment is parallel to an extension direction of the second extension segment; and the first extension segment and the second extension segment are distributed in a staggered manner in a circumferential direction of the mounting unit. Due to the bending effect of the bending segment, the connecting unit shifts from the original extension position of the first extension segment to the extension position of the second extension segment while the extension direction of the entire connecting unit remains unchanged.

### CROSS REFERENCE TO THE RELATED APPLICATIONS

This application is the national phase entry of International Application No. PCT/CN2024/071933, filed on Jan. 12, 2024, which is based upon and claims priority to Chinese Patent Application No. 202310178509.4, filed on Feb. 28, 2023, the entire contents of which are incorporated herein by reference.

### TECHNICAL FIELD

The present disclosure relates to the technical field of endoscopy, and in particular to a circuit structure, a front-end assembly, and an endoscope.

### BACKGROUND

As a commonly used medical device, the endoscope includes an operation handle and an inserting unit. The inserting unit enters the human body through a natural cavity or a surgical incision. By manipulating the lever on the operation handle, the active bending section at the distal end of the inserting unit is driven to perform attitude adjustments. The imaging module at the distal end of the inserting unit enables observation of internal tissues of the human body, assisting physicians in identifying lesion locations within the body of the patient and characterizing tissue structures at the lesion location.

The distal end of the inserting unit is provided with a front-end assembly, and the front-end assembly typically includes a circuit structure, an illumination module, and the imaging module. The illumination module and the imaging module are electrically connected to the circuit structure separately to ensure their normal operational functions. However, circuit structures in the related art are prone to fracture during operation, compromising operational stability of the front-end assembly.

### SUMMARY

In view of this, the present disclosure provides a circuit structure, a front-end assembly, and an endoscope to solve the technical problem that circuit structures in related art are prone to damage or even fracture during use.

To solve the above-mentioned problem, the present disclosure adopts the following technical solutions:

A first aspect of the present disclosure provides a circuit structure, applied to an endoscope, and including a connecting unit and a mounting unit, where the mounting unit is connected to a distal end of the connecting unit; and
the connecting unit includes a first extension segment, a bending segment, and a second extension segment connected sequentially; an extension direction of the first extension segment is parallel to an extension direction of the second extension segment; and the first extension segment and the second extension segment are distributed in a staggered manner in a circumferential direction of the mounting unit.

Furthermore, the mounting unit forms an arc-shaped plate-like structure or an arc-shaped ribbon-like structure, such that a radial inner side of the mounting unit forms an instrument tube avoidance area.

Furthermore, the mounting unit includes an imaging module mounting area; the mounting unit is provided with an illumination module mounting area located on at least one side of the imaging module mounting area; and the first extension segment is connected between the imaging module mounting area and the illumination module mounting area.

Furthermore, the connecting unit is a flexible circuit board, and the mounting unit is a flexible circuit board or a rigid circuit board.

A second aspect of the present disclosure provides a front-end assembly, including an active bending section and the above-mentioned circuit structure, where the active bending section is provided with a traction wire mounting unit; and the second extension segment corresponds to the traction wire mounting unit.

Furthermore, a distal portion of the second extension segment protrudes from a distal end surface of the active bending section.

Furthermore, the front-end assembly further includes a lens base; the lens base is connected to a distal end of the active bending section; and a distal end of the circuit structure is located in the lens base.

Furthermore, the front-end assembly further includes an intermediate adapter; the lens base is connected to the active bending section via the intermediate adapter; and a proximal portion of the first extension segment protrudes from a proximal end surface of the lens base, such that the bending segment is located in the intermediate adapter.

Furthermore, the lens base is internally provided with a wire groove, and the first extension segment is provided in the wire groove.

A third aspect of the present disclosure provides an endoscope, including the above-mentioned front-end assembly.

The technical solutions adopted in the present disclosure have the following beneficial effects:

First, in the circuit structure, the front-end assembly, and the endoscope provided by the present disclosure, due to the bending effect of the bending segment, the connecting unit shifts from the original extension position of the first extension segment to the extension position of the second extension segment. The extension direction of the entire connecting unit toward the proximal end of the endoscope remains unchanged. Thus, when the circuit structure is provided in the endoscope, the first extension segment and the second extension segment are distributed at different positions while sharing the same extension direction, thereby improving flexibility in disposing the circuit structure.

Second, the connecting unit can be a flexible circuit board. When the connecting unit is applied to the endoscope, the second extension segment is provided at a position corresponding to the traction wire mounting unit of the active bending section, such that a bendable direction of the second extension segment aligns with the bending direction of the active bending section. Thus, the present disclosure prevents damage or even fracture of the circuit structure caused by inconsistency between the bending direction of the connecting unit and the bending direction of the active bending section, ensuring stability during endoscope use.

Third, the proximal portion of the first extension segment protrudes from the proximal end surface of the lens base, and the distal portion of the second extension segment protrudes from the distal end surface of the lens base, such that the bending segment extending in the circumferential direction of the front-end assembly is entirely located within the intermediate adapter. On one hand, this design prevents the bending segment from occupying the internal space of the lens base, thereby saving the accommodation space of the lens base and facilitating its miniaturization. On the other hand, the active bending section entirely falls within the extension path of the second extension segment. That is, the bending of the active bending section cannot affect the first extension segment or the bending segment, thereby completely avoiding damage or even fracture at the connecting unit of the circuit structure.

### BRIEF DESCRIPTION OF THE DRAWINGS

To describe the technical solutions in the embodiments of the present disclosure or in the prior art more clearly, the following briefly describes the drawings required for describing the embodiments or the prior art. Apparently, the drawings in the following description show merely some embodiments of the present disclosure, and a person of ordinary skill in the art may still derive other drawings from these drawings without creative efforts.

FIG. 1 is a first structural schematic diagram of a circuit structure according to an embodiment of the present disclosure;

FIG. 2 is a second structural schematic diagram of the circuit structure according to an embodiment of the present disclosure;

FIG. 3 is a first structural schematic diagram of a front-end assembly according to an embodiment of the present disclosure;

FIG. 4 is a second structural schematic diagram of the front-end assembly according to an embodiment of the present disclosure;

FIG. 5 is an internal structural schematic diagram of a lens base according to an embodiment of the present disclosure;

FIG. 6 is a schematic diagram of the circuit structure and the lens base that are connected according to an embodiment of the present disclosure;

FIG. 7 is a third structural schematic diagram of the front-end assembly according to an embodiment of the present disclosure; and

FIG. 8 is a fourth structural schematic diagram of the front-end assembly according to an embodiment of the present disclosure.

### REFERENCE NUMERALS

110. connecting unit; 111. first extension segment; 112. bending segment; 113. second extension segment; 120. mounting unit; 200. instrument tube avoidance area; 300. active bending section; 310. traction wire mounting unit; 400. lens base; 410. wire groove; 500. intermediate adapter; and 600. traction wire.

### DETAILED DESCRIPTION OF THE EMBODIMENTS

To make the objectives, technical solutions, and advantages of the present disclosure more clearly, the technical solutions of the present disclosure will be described in detail below. Apparently, the described embodiments are only a part rather than all of the embodiments of the present disclosure. All other embodiments derived from the embodiments in the present disclosure by a person of ordinary skill in the art without creative efforts shall fall within the protection scope of the present disclosure.

It should be noted that the terms “first”, “second”, and so on in the description and claims of the present disclosure are intended to distinguish between similar objects but do not necessarily indicate a specific order or sequence. It should be understood that data used in such a way may be interchanged under appropriate circumstances such that the embodiments of the present disclosure can be implemented in an order other than those illustrated or described herein. The objects distinguished by “first”, “second”, etc. are usually of one type, and the number of objects is not limited. For example, one or more first objects may be provided. In addition, “and/or” in the specification and claims indicates at least one of the connected objects, and the character “/” generally indicates an “or” relationship between associated objects.

In the present disclosure, “proximal” and “distal” describe the relative positional relationship of the structure relative to the operator during use, facilitating the description and understanding of positional relationships between components. “Proximal” and “distal” describe relative positional relationships rather than absolute ones.

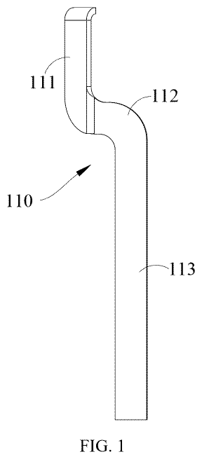

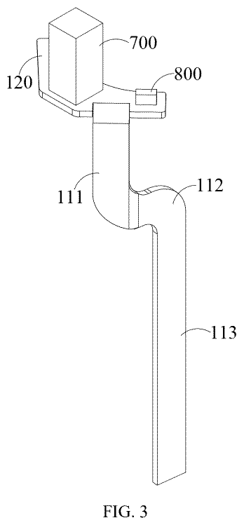

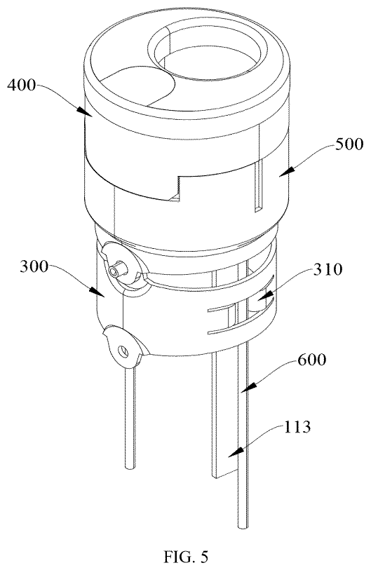

The present disclosure provides a circuit structure, a front-end assembly, and an endoscope, which are described in detail below through specific embodiments and application scenarios with reference to FIG. 1 to FIG. 8.

Please refer to FIG. 1 to FIG. 3. An embodiment of the present disclosure provides a circuit structure for an endoscope. The circuit structure includes connecting unit 110 and mounting unit 120. The mounting unit 120 is connected to a distal end of the connecting unit 110. The mounting unit 120 is configured to mount a functional module at a distal end of the endoscope, such as imaging module 700 and illumination module 800. The connecting unit 110 is configured to electrically connect the functional module to provide power, transmit data, and transmit signals.

Specifically, the connecting unit 110 includes first extension segment 111, bending segment 112, and second extension segment 113 connected sequentially. An extension direction of the first extension segment 111 is parallel to an extension direction of the second extension segment 113. The entire connecting unit 110 extends toward a proximal end of the endoscope.

A distal end of the first extension segment 111 is configured to connect the mounting unit 120 for mounting the functional module of the endoscope. The bending segment 112 is connected to a proximal end of the first extension segment 111, and is configured to change an extension position of the connecting unit 110. A distal end of the second extension segment 113 is connected to the bending segment, and a proximal end of the second extension segment 113 extends toward the proximal end of the endoscope.

That is, due to the bending effect of the bending segment 112, the connecting unit 110 shifts from the original extension position of the first extension segment 111 to the extension position of the second extension segment 113. The first extension segment 111 and the second extension segment 113 are distributed in a staggered manner in a circumferential direction of the mounting unit 120. The extension direction of the entire connecting unit 110 toward the proximal end of the endoscope remains unchanged. Thus, when the circuit structure is provided in the endoscope, the first extension segment 111 and the second extension segment 113 are distributed at different positions while sharing the same extension direction, thereby improving flexibility in disposing the circuit structure.

Meanwhile, the connecting unit 110 can adopt a flexible circuit board. When the circuit structure is provided, the bendable direction of the second extension segment 113 aligns with a bending direction of active bending section 300. When the active bending section 300 bends, the second extension segment 113 bends synchronously with the active bending section 300, effectively preventing damage or even fracture of the connecting unit 110 and ensuring stability during endoscope use.

It should be understood that in the embodiment of the present disclosure, the mounting unit 120 can be a rigid circuit board. In this case, the circuit structure can be a combination of a rigid circuit board and a flexible circuit board. Of course, the mounting unit 120 can also be a flexible circuit board. In this case, the circuit structure is an integrated flexible circuit board. The present disclosure imposes no limitation on this.

In the embodiment of the present disclosure, when the mounting unit 120 is a rigid circuit board, the mounting unit 120 forms an arc-shaped plate-like structure. When the mounting unit 120 is a flexible circuit board, the mounting unit 120 forms an arc-shaped ribbon-like structure. A radial inner side of the mounting unit 120 forms instrument tube mounting area 200. When the circuit structure is placed in the endoscope, an instrument tube of the endoscope can be provided in the instrument tube mounting area 200. In other words, the mounting unit 120 surrounds a circumferential portion of the instrument tube. The design reduces spatial occupancy of the mounting unit and the instrument tube in a radial direction of the front-end assembly of the endoscope, making the layout of the front-end assembly more compact and facilitating miniaturization of the front-end assembly.

In the embodiment of the present disclosure, the mounting unit 120 includes an imaging module mounting area and an illumination module mounting area. The illumination module mounting area is distributed on at least one side of the imaging module mounting area. That is, the mounting unit 120 can include one illumination module mounting area or two illumination module mounting areas.

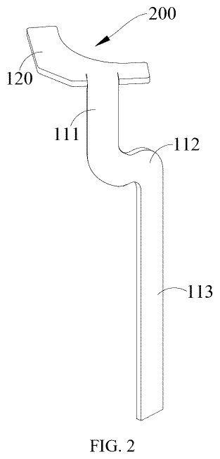

In a preferred implementation, please refer to FIG. 2. The imaging module mounting area is located at a middle of the mounting unit 120, and two illumination module mounting areas are distributed on two sides of the imaging module mounting area. Two illumination modules 800 simultaneously provide illumination, enabling the front-end assembly of the endoscope to have a large illumination range and more uniform illumination, thereby ensuring imaging quality of the imaging module 700.

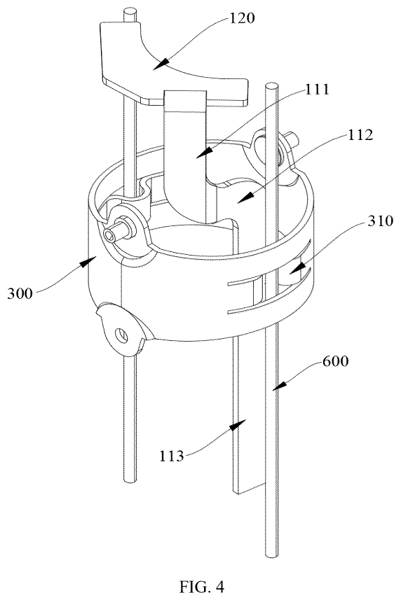

Please refer to FIG. 4 to FIG. 8. The embodiment of the present disclosure further provides a front-end assembly for an endoscope. The front-end assembly includes active bending section 300 and the circuit structure. The active bending section 300 is provided with traction wire mounting unit 310. The traction wire mounting unit 310 is configured to mount traction wire 600. By manipulating a lever on an endoscope handle, the traction wire 600 can be pulled to drive bending positional adjustment of the active bending section 300. The first extension segment 111 and the second extension segment 113 of the circuit structure extend along an axial direction of the front-end assembly, and the bending segment 112 extends along a circumferential direction of the front-end assembly.

In the embodiment of the present disclosure, the second extension segment 113 corresponds to the traction wire mounting unit 310. It should be noted that the correspondence here refers to the second extension segment 113 and the traction wire mounting unit 310 being distributed at the same position in the circumferential direction of the active bending section 300. Thus, the bending direction of the active bending section 300 aligns with the bendable direction of the second extension segment 113 of the circuit structure.

In an optional implementation, the front-end assembly can further include lens base 400. The lens base 400 is connected to a distal end of the active bending section 300. The lens base 400 provides a mounting foundation for the imaging module 700 and the illumination module 800. The mounting unit 120 of the circuit structure is provided in the lens base 400 and is electrically connected to the imaging module 700 and the illumination module 800.

Research finds that if the first extension segment 111 is connected to a position corresponding to the imaging module mounting area, the radial dimension of the lens base 400 in the distribution direction of the imaging module 700 and the instrument tube is further expanded, which is unfavorable for miniaturization of the front-end assembly.

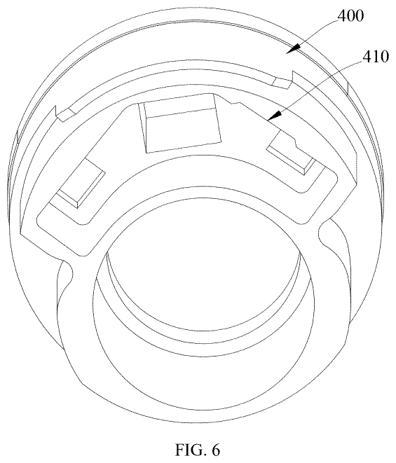

Please refer to FIG. 6. Research also finds that the space occupied by the imaging module 700 in the lens base 400 is generally larger than the space occupied by the illumination module 800 in the lens base 400. When the imaging module and the illumination module are arranged in the endoscope, a large transition area typically exists between the imaging module and the illumination module.

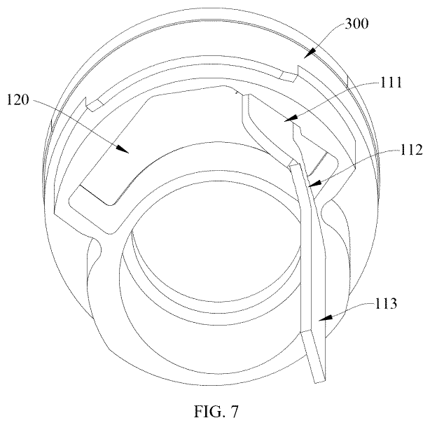

In view of the above situation, in a preferred implementation, please refer to FIG. 7. The first extension segment 111 is connected to a transition area between the imaging module mounting area and the illumination module mounting area. Thus, the connecting unit 110 is offset to one side of the distribution direction of the imaging module 700 and the instrument tube. The design does not require expanding an accommodation area for disposing the connecting unit 110 in the distribution direction of the imaging module 700 and the instrument tube, optimizes the spatial layout of the circuit structure, and facilitates miniaturization of the front-end assembly of the endoscope.

Meanwhile, the design also avoids significant deformation of the imaging module mounting area or the illumination module mounting area caused by pulling from the connecting unit 110, thereby preventing separation of the imaging module 700 from the imaging module mounting area or separation of the illumination module 800 from the illumination module mounting area. This ensures connection stability between the mounting unit 120 and the imaging module 700 as well as the illumination module 800.

In a further technical solution, please continue to refer to FIG. 6. Wire groove 410 corresponding to the first extension segment 111 is provided in the lens base 400. The first extension segment 111 is provided in the wire groove 410. The wire groove 410 constrains the first extension segment 111 to prevent circumferential movement of the first extension segment 111 in the lens base 400, thereby preventing the second extension segment 113 and the traction wire mounting unit 310 from remaining distributed at the same position in the circumferential direction of the active bending section 300.

In the embodiment of the present disclosure, the front-end assembly can further include intermediate adapter 500. The intermediate adapter 500 is a tubular structure. The lens base 400 is connected to the active bending section 300 via the intermediate adapter 500.

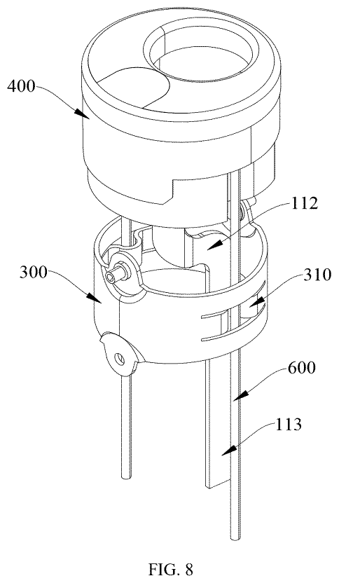

In a further technical solution, please refer to FIG. 8. A proximal portion of the first extension segment 111 protrudes from a proximal end surface of the lens base 400, and a distal portion of the second extension segment 113 protrudes from a distal end surface of the active bending section 300. Thus, the bending segment 112 extending along the circumferential direction of the front-end assembly is entirely located within the intermediate adapter 500. On one hand, the design avoids occupation of the accommodation space of the lens base 400 by the bending segment 112 extending along the circumferential direction of the front-end assembly, saving the accommodation space of the lens base 400. On the other hand, the active bending section 300 entirely falls within an extension path of the second extension segment 113. That is, bending of the active bending section 300 can only affect the second extension segment 113 and cause its passive bending, without affecting the first extension segment 111 and the bending segment 112. The design completely prevents damage or even fracture of the connecting unit 110 of the circuit structure.

The embodiment of the present disclosure further provides an endoscope, including the above-mentioned front-end assembly.

The endoscope in the embodiment of the present disclosure may be a gastroscope, enteroscope, laryngoscope, fiber bronchoscope, etc. The present disclosure imposes no specific limitations on the type of the endoscope.

It should be noted that terms “including”, “comprising” or any other variants thereof are intended to cover non-exclusive inclusion such that a process, method, article, or apparatus including a series of elements includes not only those elements but also other elements not explicitly listed, or elements inherent to such a process, method, article, or apparatus. Without further limitation, an element qualified by the phrase “including a . . . ” does not exclude the presence of an additional identical element in the process, method, article, or apparatus including the element. Additionally, it should be noted that the scope of the method and apparatus in the embodiments of the present disclosure is not limited to performing functions in the order explicitly shown or discussed. Functions may alternatively be executed substantially concurrently or in reverse order. For example, the method may involve steps performed in sequences differing from the stated order, and steps may also be added, omitted, or combined. Furthermore, features described with reference to specific examples may be incorporated into other embodiments.

The above are merely specific implementations of the present disclosure, and the protection scope of the present disclosure is not limited thereto. Any modification or replacement easily conceived by those skilled in the art within the technical scope of the present disclosure should fall within the protection scope of the present disclosure.

What is claimed is:

1-10. (canceled)

11. A circuit structure, applied to a front-end assembly of an endoscope, and comprising a connecting unit and a mounting unit, wherein the mounting unit is connected to a distal end of the connecting unit;
the connecting unit comprises a first extension segment, a bending segment, and a second extension segment connected sequentially; an extension direction of the first extension segment is parallel to an extension direction of the second extension segment; and the bending segment extends along a circumferential direction of the front-end assembly, such that the first extension segment and the second extension segment are distributed in a staggered manner in a circumferential direction of the mounting unit; and
the mounting unit forms an arc-shaped plate-like structure or an arc-shaped ribbon-like structure, such that a radial inner side of the mounting unit forms an instrument tube avoidance area; the mounting unit comprises an imaging module mounting area; the mounting unit is provided with an illumination module mounting area located on at least one side of the imaging module mounting area; and the first extension segment is connected between the imaging module mounting area and the illumination module mounting area.

12. The circuit structure according to claim 11, wherein the connecting unit is a flexible circuit board, and the mounting unit is a flexible circuit board or a rigid circuit board.

13. A front-end assembly, comprising an active bending section and the circuit structure according to claim 11, wherein the active bending section is provided with a traction wire mounting unit; and the second extension segment corresponds to the traction wire mounting unit.

14. The front-end assembly according to claim 13, wherein a distal portion of the second extension segment protrudes from a distal end surface of the active bending section.

15. The front-end assembly according to claim 14, wherein the front-end assembly further comprises a lens base; the lens base is connected to a distal end of the active bending section; and a distal end of the circuit structure is located in the lens base.

16. The front-end assembly according to claim 15, wherein the front-end assembly further comprises an intermediate adapter; the lens base is connected to the active bending section via the intermediate adapter; and a proximal portion of the first extension segment protrudes from a proximal end surface of the lens base, such that the bending segment is located in the intermediate adapter.

17. The front-end assembly according to claim 15, wherein the lens base is internally provided with a wire groove, and the first extension segment is provided in the wire groove.

18. An endoscope, comprising the front-end assembly according to claim 13.

19. The front-end assembly according to claim 11, wherein in the circuit structure, the connecting unit is a flexible circuit board, and the mounting unit is a flexible circuit board or a rigid circuit board.

20. The endoscope according to claim 18, wherein in the front-end assembly, a distal portion of the second extension segment protrudes from a distal end surface of the active bending section.

21. The endoscope according to claim 20, wherein in the front-end assembly, the front-end assembly further comprises a lens base; the lens base is connected to a distal end of the active bending section; and a distal end of the circuit structure is located in the lens base.

22. The endoscope according to claim 21, wherein in the front-end assembly, the front-end assembly further comprises an intermediate adapter; the lens base is connected to the active bending section via the intermediate adapter; and a proximal portion of the first extension segment protrudes from a proximal end surface of the lens base, such that the bending segment is located in the intermediate adapter.

23. The endoscope according to claim 21, wherein in the front-end assembly, the lens base is internally provided with a wire groove, and the first extension segment is provided in the wire groove.
